(19) United States
(12) Patent Application Publication
(10) Pub. No.: US 2008/0213892 A1
(43) Pub. Date: Sep. 4, 2008

(54) SELF-RENEWAL OF NEURAL STEM CELLS IS PROMOTED BY WNT PROTEINS

(75) Inventors: Roeland Nusse, Stanford, CA (US); M. Yashar S. Kalani, Irvine, CA (US); Samuel Cheshier, Palo Alto, CA (US)

Correspondence Address: BOZICEVIC, FIELD & FRANCIS LLP, 1900 UNIVERSITY AVENUE, SUITE 200, EAST PALO ALTO, CA, 94303, US

(73) Assignee: The Board of Trustees of the Leland Stanford Junior University

(21) Appl. No.: 12/041,538

(22) Filed: Mar. 3, 2008

Related U.S. Application Data
(60) Provisional application No. 60/904,596, filed on Mar. 2, 2007.

(51) Int. Cl. C12N 5/06

(52) U.S. Cl. 435/377

(57) ABSTRACT

Mammalian neural progenitor or stem cells are expanded in vitro by culture in the presence of one or more wnt polypeptides. The expanded cells substantially maintain their original phenotype including the ability to give rise to multiple types of differentiated cells.

### INTRODUCTION

Adult neurogenesis is restricted to two regions of the central nervous system: the subventricular zone of the lateral ventricle, and the subgranular zone of the hippocampal dentate gyrus (Lie et al., 2005). Hippocampal neurogenesis occurs throughout life and the balance of neuronal loss and birth is essential in generating the plasticity necessary for new memory formation. The generation of new neurons within the hippocampus is mediated by proliferating neural stem/progenitor cells that are exquisitely sensitive to local signaling. Stem cells represent the most immature cell necessary for neurogenesis. These cells give rise to more restricted precursors or progenitor cells and ultimately these progenitors differentiate into new functional neurons. These cells produce neurons in response to signals received from surrounding cells as well as humoral signals from circulating hormones, cytokines, and growth factors.

In the embryonic setting, neurogenesis is likely to be less restricted anatomically; but, in both cases, signals provided by the microenvironment regulate the maintenance, proliferation and neuronal fate commitment of the local stem cell populations. While much effort has been devoted to understand neurogenesis in both the adult and embryonic settings, the identity of the signals regulating these processes is largely unknown.

Wnt signaling and Wnt proteins have been implicated in the maintenance of certain stem cells. One example of the role of Wnt proteins is in the gut, where in the crypt of the colon the loss of transcription factor TCF4 leads to depletion of stem cells (van de Wetering et al., 2002; Korinek et al., 1998). Alternatively, loss of the tumor suppressor APC or gain of β-catenin activity leads to excess of stem cells and colon cancer (Bienz and Clevers, 2000).

For many purposes, there is an interest in being able to expand stem and progenitor cells in culture. However, it is not simply a matter of maintaining cell viability for the stem cells, but also of ensuring that the stem cells increase in numbers without losing their distinctive phenotype. While a substantial increase in cell number can be obtained with many cultures, they do not always provide for expanded number of cells that retain a capacity for long term repopulation.

There continues to be a strong demand for improvements in the in vitro culture of stem cells and progenitor cells, as well as methods for their manipulation in vivo. The present invention addresses this need. Given the important role of Wnt proteins as potential regulators of stem cell populations to self-renew, proliferate or differentiate, we set out to understand the role of the Wnt signaling pathway in the fate decision of embryonic neural stem cells.

### Related Publications

Parkin N., Kitajewski J. and Varmus H. (1993) Activity of Wnt-1 as a transmembrane protein. Genes & Dev., 7, 2181-2193. Lee S. M., Tole S., Grove E. and McMahon A. P. (2000) A local Wnt-3a signal is required for development of the mammalian hippocampus. Development, 127, 457-467. Lie D. C., Colamarino S. A. Song, H. Desire L., Mira H., Consiglio A., Lein E. S., Jessberger S., Lansford H., Dearie E. A., and Gage F. H. (2005) Wnt signalling regulates adult hippocampal Neurogenesis. Nature, 437, 1370-1375. Chenn A. and Walsh C. A. (2002) Regulation of cerebral cortial size by control of cell cycle exit in neural precursors. Science, 297, 365-369.

### SUMMARY OF THE INVENTION

Methods are provided for the expansion of neural progenitor or stem cells in vitro, whereby the cells retain their pluripotential phenotype after expansion. The cells are cultured in the presence of a wnt activating agent, e.g. a wnt polypeptide. The alteration in wnt signaling provides for cultures that maintain an undifferentiated phenotype in vitro. The expanded cell populations are useful as a source of stem cells, e.g. to reconstitute function in a host that is deficient in a particular cell lineage or lineages.

A Wnt responsive population of cells from the embryo or from adult tissues can be isolated by flow cytometry. These cells can be cultured in vitro in a serum free condition. The addition of Wnt protein to the cultures causes a clonogenic outgrowth of neural stem cells. In vitro manipulation of these cells results in colonies that are multipotential, and can form the three cell types of the central nervous system. Blocking Wnt signaling pathway results in a depletion of stem cell populations. The data demonstrate that Wnt proteins can be used in an in vitro setting for the manipulation and engineering of cells useful for cell-based therapeutics.

### BRIEF DESCRIPTION OF THE DRAWINGS

FIG. 1A-C. A. A representative limiting dilution analysis of the affect of Wnt and Dkk on neural stem cells. Wnt treatment expands the neural progenitor population at all cell densities. Dkk depletes the progenitor population at all cell densities. B. Average Expansion/Depletion of progenitors averaged across 5 experiments. C. Fold expansion of spheres # relative to control treated culture. Wnt enhances colony-forming ability whereas Dkk inhibits it.

FIGS. 2A-2D. A. Primary sphere geometry-Sphere geometry of control, Wnt3a, and Dkk treated cultures. Wnt-treated cultures produce smaller colonies relative to control and Dkk. B. Primary control treated spheres. C. Primary Wnt treated spheres. D. Primary Dkk treated spheres.

FIG. 3. Wnt promotes self-renewal of the neural progenitor whereas Dkk inhibits it.

FIGS. 4A-C. A. Secondary control treated spheres. B. Secondary Wnt treated spheres. C. Secondary sphere geometry-Sphere geometry of Control and Wnt3a secondary spheres. Results pooled from 3 experiments.

FIGS. 5A-B. A. Cells treated with the Wnt protein produce all three cell types of the CNS. B. Cells treated with the Wnt protein produce all three cell types of the CNS.

FIG. 6. FACS analysis shows a small subpopulation of the cells from the E14.5 telencephalon is endogenously activated by the Wnt pathway. This population corresponds to 9% of all cells.

### DESCRIPTION OF THE SPECIFIC EMBODIMENTS

Methods are provided for the expansion of neural progenitor or stem cells in vitro. In some embodiments of the invention, the Wnt protein is a mammalian protein, including, without limitation, human Wnt proteins, e.g. Wnt3A.

### DEFINITIONS

Before the present methods are described, it is to be understood that this invention is not limited to particular methods described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges encompassed within the invention, subject to any specifically excluded limit in the stated range.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a microsphere” includes a plurality of such microspheres and reference to “the stent” includes reference to one or more stents and equivalents thereof known to those skilled in the art, and so forth.

Wnt protein. Wnt proteins form a family of highly conserved secreted signaling molecules that regulate cell-to-cell interactions during embryogenesis. The terms “Wnts” or “Wnt gene product” or “Wnt polypeptide” when used herein encompass native sequence Wnt polypeptides, Wnt polypeptide variants, Wnt polypeptide fragments and chimeric Wnt polypeptides. In some embodiments of the invention, the Wnt protein comprises palmitate covalently bound to a cysteine residue.

A “native sequence” polypeptide is one that has the same amino acid sequence as a Wnt polypeptide derived from nature. Such native sequence polypeptides can be isolated from cells producing endogenous Wnt protein or can be produced by recombinant or synthetic means. Thus, a native sequence polypeptide can have the amino acid sequence of, e.g. naturally occurring human polypeptide, murine polypeptide, or polypeptide from any other mammalian species, or from non-mammalian species, e.g. Drosophila, C. elegans, and the like.

The term “native sequence Wnt polypeptide” includes human and murine Wnt polypeptides. Human wnt proteins include the following: Wnt 1, Genbank reference NP_{—}005421.1; Wnt 2, Genbank reference NP_{—}003382.1, which is expressed in brain in the thalamus, in fetal and adult lung and in placenta; two isoforms of Wnt 2B, Genbank references NP_{—}004176.2 and NP_{—}078613.1. Isoform 1 is expressed in adult heart, brain, placenta, lung, prostate, testis, ovary, small intestine and colon. In the adult brain, it is mainly found in the caudate nucleus, subthalamic nucleus and thalamus. Also detected in fetal brain, lung and kidney. Isoform 2 is expressed in fetal brain, fetal lung, fetal kidney, caudate nucleus, testis and cancer cell lines. Wnt 3 and Wnt3A play distinct roles in cell-cell signaling during morphogenesis of the developing neural tube, and have the Genbank references NP_{—}110380.1 and X56842. Wnt3A is expressed in bone marrow. Wnt 4 has the Genbank reference NP_{—}110388.2. Wnt 5A and Wnt 5B have the Genbank references NP_{—}003383.1 and AK013218. Wnt 6 has the Genbank reference NP_{—}006513.1; Wnt 7A is expressed in placenta, kidney, testis, uterus, fetal lung, and fetal and adult brain, Genbank reference NP_{—}004616.2. Wnt 7B is moderately expressed in fetal brain, weakly expressed in fetal lung and kidney, and faintly expressed in adult brain, lung and prostate, Genbank reference NP_{—}478679.1. Wnt 8A has two alternative transcripts, Genbank references NP_{—}114139.1 and NP 490645.1. Wnt 8B is expressed in the forebrain, and has the Genbank reference NP_{—}003384.1. Wnt 10A has the Genbank reference NP_{—}079492.2. Wnt 10B is detected in most adult tissues, with highest levels in heart and skeletal muscle. It has the Genbank reference NP_{—}003385.2. Wnt 11 is expressed in fetal lung, kidney, adult heart, liver, skeletal muscle, and pancreas, and has the Genbank reference NP_{—}004617.2. Wnt 14 has the Genbank reference NP_{—}003386.1. Wnt 15 is moderately expressed in fetal kidney and adult kidney, and is also found in brain. It has the Genbank reference NP_{—}003387.1. Wnt 16 has two isoforms, Wnt-16a and Wnt-16b, produced by alternative splicing. Isoform Wnt-16B is expressed in peripheral lymphoid organs such as spleen, appendix, and lymph nodes, in kidney but not in bone marrow. Isoform Wnt-16a is expressed at significant levels only in the pancreas. The Genbank references are NP_{—}057171.2 and NP_{—}476509.1.

The term “native sequence Wnt protein” includes the native proteins with or without the initiating N-terminal methionine (Met), and with or without the native signal sequence. The native sequence human and murine Wnt polypeptides known in the art are from about 348 to about 389 amino acids long in their unprocessed form reflecting variability (particularly at the poorly conserved amino-terminus and several internal sites), contain 21 conserved cysteines, and have the features of a secreted protein. The molecular weight of a Wnt polypeptide is about 38-42 kD.

A “variant” polypeptide means a biologically active polypeptide as defined below having less than 100% sequence identity with a native sequence polypeptide. Such variants include polypeptides wherein one or more amino acid residues are added at the N- or C-terminus of, or within, the native sequence; from about one to forty amino acid residues are deleted, and optionally substituted by one or more amino acid residues; and derivatives of the above polypeptides, wherein an amino acid residue has been covalently modified so that the resulting product has a non-naturally occurring amino acid. Ordinarily, a biologically active Wnt variant will have an amino acid sequence having at least about 90% amino acid sequence identity with a native sequence Wnt polypeptide, preferably at least about 95%, more preferably at least about 99%.

A “chimeric” Wnt polypeptide is a polypeptide comprising a Wnt polypeptide or portion (e.g., one or more domains) thereof fused or bonded to heterologous polypeptide. The chimeric Wnt polypeptide will generally share at least one biological property in common with a native sequence Wnt polypeptide. Examples of chimeric polypeptides include immunoadhesins, combine a portion of the Wnt polypeptide with an immunoglobulin sequence, and epitope tagged polypeptides, which comprise a Wnt polypeptide or portion thereof fused to a “tag polypeptide”. The tag polypeptide has enough residues to provide an epitope against which an antibody can be made, yet is short enough such that it does not interfere with biological activity of the Wnt polypeptide. Suitable tag polypeptides generally have at least six amino acid residues and usually between about 6-60 amino acid residues.

A “functional derivative” of a native sequence Wnt polypeptide is a compound having a qualitative biological property in common with a native sequence Wnt polypeptide. “Functional derivatives” include, but are not limited to, fragments of a native sequence and derivatives of a native sequence Wnt polypeptide and its fragments, provided that they have a biological activity in common with a corresponding native sequence Wnt polypeptide. The term “derivative” encompasses both amino acid sequence variants of Wnt polypeptide and covalent modifications thereof.

Biologically Active Wnt The methods of the present invention provide for Wnt compositions that are active when contacted with a cell. One may determine the specific activity of a Wnt protein in a composition by determining the level of activity in a functional assay, quantitating the amount of Wnt protein present in a non-functional assay, e.g. immunostaining, ELISA, quantitation on coomasie or silver stained gel, etc., and determining the ratio of biologically active Wnt to total Wnt.

Stem cell: The term stem cell is used herein to refer to a mammalian cell that has the ability both to self-renew, and to generate differentiated progeny (see Morrison et al. (1997) Cell 88:287-298). Generally, stem cells also have one or more of the following properties: an ability to undergo asynchronous, or symmetric replication, that is where the two daughter cells after division can have different phenotypes; extensive self-renewal capacity; capacity for existence in a mitotically quiescent form; and clonal regeneration of all the tissue in which they exist, for example the ability of hematopoietic stem cells to reconstitute all hematopoietic lineages. “Progenitor cells” differ from stem cells in that they typically do not have the extensive self-renewal capacity, and often can only regenerate a subset of the lineages in the tissue from which they derive, for example only lymphoid, or erythroid lineages in a hematopoietic setting.

Stem cells may be characterized by both the presence of markers associated with specific epitopes identified by antibodies and the absence of certain markers as identified by the lack of binding of specific antibodies. Stem cells may also be identified by functional assays both in vitro and in vivo, particularly assays relating to the ability of stem cells to give rise to multiple differentiated progeny.

The stem cells of interest are typically mammalian, where the term refers to any animal classified as a mammal, including humans, domestic and farm animals, and zoo, laboratory, sports, or pet animals, such as dogs, horses, cats, cows, mice, rats, rabbits, etc. Preferably, the mammal is human.

The cells which are employed may be fresh, frozen, or have been subject to prior culture. They may be fetal, neonate, adult. The manner in which the stem cells are separated from other cells of the hematopoietic or other lineage is not critical to this invention. A substantially homogeneous population of stem or progenitor cells may be obtained by selective isolation of cells free of markers associated with differentiated cells, while displaying epitopic characteristics associated with the stem cells.

Neural Stem Cells. Neurogenesis, i.e., the production of new neurons, has until recently been considered to occur only during the embryonic and early postnatal periods and to have no significant role in the adult brain. In recent years this doctrine has had to be revised to acknowledge that at least limited neurogenesis occurs normally in selected regions of adult mammalian brain and that neurogenesis may be stimulated in these as well as other regions in response to injury. This modified view has come about in part because of technical improvements in methods available for labeling dividing cells as well as the availability of a broad array of cell-type-specific markers that allowed cells to be identified on other than morphological grounds. This concept has also been bolstered by the finding that a resident population of neural progenitor cells exists in adult brain, obviating the need for mature neurons to become mitotic.

During development of the central nervous system and peripheral nervous system, neural stem cells proliferate and give rise to transiently dividing progenitor cells that eventually differentiate into the cell types that compose the adult brain. Cells which are derived from the neural tube give rise to neurons and glia of the CNS, while cells derived from the neural crest give rise to the cells of the peripheral nervous system (PNS).

Neural stem/progenitor cells have been described in the art, and their use in a variety of therapeutic protocols has been widely discussed. For example, inter alia, U.S. Pat. No. 6,638,501, Bjornson et al.; U.S. Pat. No. 6,541,255, Snyder et al.; U.S. Pat. No. 6,498,018, Carpenter; U.S. Patent Application 20020012903, Goldman et al.; Palmer et al. (2001) Nature 411(6833):42-3; Palmer et al. (1997) Mol Cell Neurosci. 8(6):389-404; Svendsen et al. (1997) Exp. Neurol. 148(1):135-46 and Shihabuddin (1999) Mol Med Today. 5(11):474-80. Methods for isolation and culture of neural stem cells are known in the art, e.g. see U.S. Pat. No. 6,777,233; U.S. Pat. No. 6,497,872, and US Patent application 20030143737A1, each herein specifically incorporated by reference.

Human CNS neural stem cells, when maintained in a mitogen-containing (typically epidermal growth factor or epidermal growth factor plus basic fibroblast growth factor), serum-free culture medium, have previously been found to grow in suspension culture to form aggregates of cells known as “neurospheres”. Upon removal of the mitogen(s) and provision of a substrate, the stem cells differentiate into neurons, astrocytes and oligodendrocytes.

Neural stem and progenitor cells can participate in aspects of normal development, including migration along well-established migratory pathways to disseminated CNS regions, differentiation into multiple developmentally- and regionally-appropriate cell types in response to microenvironmental cues, and non-disruptive, non-tumorigenic interspersion with host progenitors and their progeny. Human NSCs are capable of expressing foreign transgenes in vivo in these disseminated locations. As such, these cells find use in the treatment of a variety of conditions, including traumatic injury to the spinal cord, brain, and peripheral nervous system; treatment of degenerative disorders.

The similarities between neural stem cells in the central and peripheral nervous system also indicate that these methods are useful in augmenting neural tissue repair in the peripheral nervous system, where local inflammation may prevent optimum healing or restoration of innervation by virtue of neural stem/progenitor cell dysfunction. Such diseases or injury may include nerve injury due to trauma, surgery, cancer, or immune disease such as multiple sclerosis, ALS, or other motor neuron disease where endogenous or grafted progenitor/stem cells are influenced by immune mechanisms.

Culture medium: The stem or progenitor cells are grown in vitro in an appropriate liquid nutrient medium. Generally, the seeding level will be at least about 10 cells/ml, more usually at least about 100 cells/ml and generally not more than about 10⁵ cells/ml, usually not more than about 10⁴ cells/ml.

Various media are commercially available and may be used, including Neurobasal-A medium; Ex vivo serum free medium; Dulbecco's Modified Eagle Medium (DMEM), RPMI, Iscove's medium, etc. The medium may be supplemented with serum or with defined additives. Appropriate antibiotics to prevent bacterial growth and other additives, such as 1% N2 supplement (Gibco), 2% B27-without vitamin-A supplement (Gibco), 60 mg/ml N-acetylcysteine (Sigma), non-essential amino acids, and penicillin/streptomycin (Biowhittaker); pyruvate (0.1-5 mM), glutamine (0.5-5 mM), 2-mercaptoethanol (1−10×10⁻⁵ M) may also be included.

Culture in serum-free medium is of particular interest. The medium may be any conventional culture medium, generally supplemented with additives such as iron-saturated transferrin, human serum albumin, soy bean lipids, linoleic acid, cholesterol, alpha thioglycerol, crystalline bovine hemin, etc., that allow for the growth of neural cells. The medium for expansion may be supplemented with bFGF; EGF; etc.

Where differentiation cultures are desired, the medium may include N-acetylcysteine, brain-derived neurotrophic factor; glial-derived neurotrophic factor; EFG and FGF on laminin-coated chamber slides.

Neural Conditions of Interest CNS disorders encompass numerous conditions, such as neurodegenerative diseases (e.g. Alzheimer's and Parkinson's), acute brain injury (e.g. stroke, head injury, cerebral palsy) and a large number of CNS dysfunctions (e.g. depression, epilepsy, and schizophrenia). In recent years neurodegenerative disease has become an important concern due to the expanding elderly population which is at greatest risk for these disorders. These diseases, which include Alzheimer's Disease, Huntington's Disease, Amyotrophic Lateral Sclerosis, and Parkinson's Disease, have been linked to the degeneration of neural cells in particular locations of the CNS, leading to the inability of these cells or the brain region to carry out their intended function.

In addition to neurodegenerative diseases, acute brain injuries often result in the loss of neural cells, the inappropriate functioning of the affected brain region, and subsequent behavior abnormalities. In addition to cell loss, an individual may suffer from an abnormal functioning of existing neural cells. This may be due to inappropriate firing of neurons, or the abnormal synthesis, release, and processing of neurotransmitters. These dysfunctions may be the result of well studied and characterized disorders such as depression and epilepsy, or less understood disorders such as neurosis and psychosis. Other forms of neurological impairment can occur as a result of neural degeneration, such as amyotrophic lateral sclerosis and cerebral palsy, or as a result of CNS trauma, such as stroke and epilepsy.

Degeneration in a brain region known as the basal ganglia can lead to diseases with various cognitive and motor symptoms, depending on the exact location. The basal ganglia consists of many separate regions, including the striatum (which consists of the caudate and putamen), the globus pallidus, the substantia nigra, substantia innominate, ventral palladium, nucleus basalis of Meynert, ventral tegmental area and the subthalamic nucleus.

In the case of Alzheimer's Disease, there is a profound cellular degeneration of the forebrain and cerebral cortex. In addition, upon closer inspection, a localized degeneration in an area of the basal ganglia, the nucleus basalis of Meynert, appears to be selectively degenerated. This nucleus normally sends cholinergic projections to the cerebral cortex which are thought to participate in cognitive functions including memory.

Many motor deficits are a result of degeneration in the basal ganglia. Huntington's Chorea is associated with the degeneration of neurons in the striatum, which leads to involuntary jerking movements in the host. Degeneration of a small region called the subthalamic nucleus is associated with violent flinging movements of the extremities in a condition called ballismus, while degeneration in the putamen and globus pallidus is associated with a condition of slow writhing movements or athetosis. In the case of Parkinson's disease, degeneration is seen in another area of the basal ganglia, the substantia nigra par compacta. This area normally sends dopaminergic connections to the dorsal striatum which are important in regulating movement. Therapy for Parkinson's disease has centered upon restoring dopaminergic activity to this circuit.

### Expansion of Stem/Progenitor Cells

A population of cells comprising neural progenitor and/or stem cells is cultured in vitro in the presence of one or more wnt proteins. A wnt polypeptide will be present in the medium at a concentration of at least about 1 ng/ml, usually at least about 10 ng/ml; 50 ng/ml, 100 ng/ml, 250 ng/ml or more. In some embodiments the wnt polypeptide is wnt 3a, wnt 5, or a combination thereof. The wnt polypeptides may be present as a free polypeptide, or may be provided where the Wnt protein is inserted in the non-aqueous phase of a lipid structure, e.g. in the surface of a liposome, micelle, lipid raft, etc., in an emulsion, and the like. Alternatively, the wnt polypeptides may be coated on a bead, e.g. heparin coated bead, etc.

The wnt polypeptide is provided at a concentration sufficient to maintain or increase the number of assayable stem or progenitor cells in the culture. The number of assayable progenitor cells may be demonstrated by a number of assays. After one week the progenitor cell cloning efficiency will usually be at least about 75% that of the starting cell population, more usually 100% that of the starting cell population, and may be as high as 200% that of the starting cell population.

Following the initial period, there is an increased expansion, where the number of assayable cells having the functional phenotype of the initial cell population can increase from about 5 to about 100 fold or more. After this time, the cells can remain in cycle, and expansion is limited primarily by considerations of space. The cells can be frozen using conventional methods at any time, usually after the first week of culture.

After seeding the culture medium, the culture medium is maintained under conventional conditions for growth of mammalian cells, generally about 37° C. and 5% CO₂ in 100% humidified atmosphere. Fresh media may be conveniently replaced, in part, by removing a portion of the media and replacing it with fresh media. Various commercially available systems have been developed for the growth of mammalian cells to provide for removal of adverse metabolic products, replenishment of nutrients, and maintenance of oxygen. By employing these systems, the medium may be maintained as a continuous medium, so that the concentrations of the various ingredients are maintained relatively constant or within a predescribed range. Such systems can provide for enhanced maintenance and growth of the subject cells using the designated media and additives.

These cells may find various applications for a wide variety of purposes. The cell populations may be used for screening various additives for their effect on growth and the mature differentiation of the cells. In this manner, compounds which are complementary, agonistic, antagonistic or inactive may be screened, determining the effect of the compound in relationship with one or more of the different cytokines. The populations of expanded cells may be employed as grafts for transplantation.

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. Due to biological functional equivalency considerations, changes can be made in protein structure without affecting the biological action in kind or amount. All such modifications are intended to be included within the scope of the appended claims.

### EXPERIMENTAL

### Example 1

### Materials and Methods

Isolation of CNS stem cells In all cases, tissue was removed from E14.5 forebrains of mice, and suspended after dissociation in culture medium: Neurobasal-A, penicillin/streptomycin (BioWhittaker, Walkersville, Md.), and 400 units/mL deoxyribonuclease type 1 (DNAse1, Worhtington Chemicals), 2.5 units/ml papin (Worthington Chemicals), and 2 mg/ml dispase (Roche Chemicals). After centrifuging, the cells were triturated with pipettes of various calibers, filtered through nylon screen (40 micron filter (BD Falcon), counted by hemocytometer, and plated.

Cell culture Non-adherent cultures of CNS stem cells were performed by plating cells on ultra nonadherent plates (Corning Incorporated, Corning, N.Y.). In all cases the culture medium was based on a Neurobasal-A medium. The medium was supplemented with 20 ng/ml recombinant human bFGF (R&D Systems, Minneapolis, Minn.), 20 ng/ml recombinant EGF(R&D Systems, Minneapolis, Minn.), 1% N2 supplement (Gibco), 2% B27-without vitamin-A supplement (Gibco), 60 mg/ml N-acetylcysteine (Sigma), non-essential amino acids, and penicillin/streptomycin (Biowhittaker). All cultures were maintained at 37° C. in 6% CO₂/balance air. Cultures were fed every other day. In some cases, sorted and unsorted cells were plated into 96-well plates to evaluate directly the frequency of precursors that initiate neurosphere cultures. Plates were scored for neurosphere growth at 10-14 days. Linear regression analysis of the proportion of negative wells at each cell concentration was used to determine the frequency of NS-IC (Uchida et al. 2000).

Differentiation Conditions Neurosphere cells were harvested, and cultured with differentiation medium [Nacetylcsteine, brain-derived neurotrophic factor (10 ng/ml) glial-derived neurotrophic factor (10 ng/ml), EFG and FGF on laminin-coated chamber slides. After 1-2 weeks, chamber slides were fixed with 4% paraformaldehyde in PBS and stained to detect differentiation into neurons oligodendrocytes, and astrocytes, and retention of any progenitors with mAbs against double cortin (1:800; Chemicon), NG2 (1:500, Chemicon), glial fibrillary acidic protein (1:500; Chemicon) and musashi (1:800, Chemicon). An anti β-galactosidase antibody was used to detect the activation of the Wnt pathway (1:500-1000, Promega). In all cases, cells were counter stained for 10 min at RT with 10 mg/ml DAPI (Sigma D-8417) to visualize nuclei.

Protein Purification Wnt3a Purification-Wnt3a protein was purified from 6.5 L of media conditioned by mouse L-cells stably overexpressing mouse Wnt3a (American Type Culture Collection (ATCC) CRL-2814) created in the lab as previously described (Willert et al. 2003). Peak fractions were then further purified by heparin affinity chromatography in some purification as previously described. Typical yields of Wnt3a protein following final heparin affinity step and concentration range from 50-100 ng/uL as assessed by coomassie staining. Dkk Purification-Dkk1c protein was purified from 1 L of media conditioned by 293 cells stably overexpressing mouse Dkk1c protein as described in Fedi et al, 1999. Typical yields of Dkk1c protein following purification are 50-200 ng/uL as assessed by Bradford quantification.

x-Gal Staining CNS colonies were stained by incubating them with x-Gal staining reagent overnight at 37° C. prior to fixation in 4% paraformaldehyde for 20 min at 4° C.

Embryonic Tissue Preparation E14.5 animals from Wnt-reporter mice were fixed in 4% paraformaldehyde for 5 days followed by embedding in cryoprotectant (1×PBS with 25% glycerin and 25% ethylene glycol at pH 6.7 and stored at −20° C.). They were subsequently sectioned and stained with the same antibody concentrations as described above.

Adult Tissue Preparation Six to 8 week adults were perfused with PBS/10 mM EDTA and 4% paraformaldehyde, their brains were removed and fixed for 24 hours in 4% paraformaldehyde/30% sucrose and sectioned on a microtome at 30 microns per sections.

Cell Sorting Single cell suspensions were generated as described in the isolation of neural stem cell section. Cells were incubated with the FACS Blue lacZ β-Galactosidase Detection kit (Marker Gene Technologies, Inc.) at a dilution of 1:50 and incubated at 37° C. for 30 mins prior to sorting on a Beckman Aria FACS-sorter.

### Results

The Colony Forming Assay—Although well established in the hematopoetic system, the colony-forming assay for the central nervous system is less well established. In order to gain insight into the role of any growth factor on the ability of cells to self-renew, proliferate or differentiate at the clonal level, one must first identify the cell density that results in clonal expansion and not aggregation. Currently, the majority of the studies in the literature have failed to identify such density. We set out to define a cell density that ensure clonality of cultures by mixing cells from a β-actin GFP and a non-GFP mouse at several cell densities. At cell densities of greater than 1 cell/μl we observe colonies that contain both green and white cells suggesting these colonies are the result of aggregation of GFP and non-GFP cells. At cell densities equal to or less than 1 cell/μl we observe colonies consisting of only GFP or non-GFP cells suggesting these colonies are more likely to be the result of clonal expansion. This observation does not preclude the fact that two non-GFP or two GFP cells could not have migrated towards one another and aggregated, but it makes this event exceedingly less likely. Colonies generated by this study were imaged using a confocal microscope; these colonies exhibit less than 1% contamination.

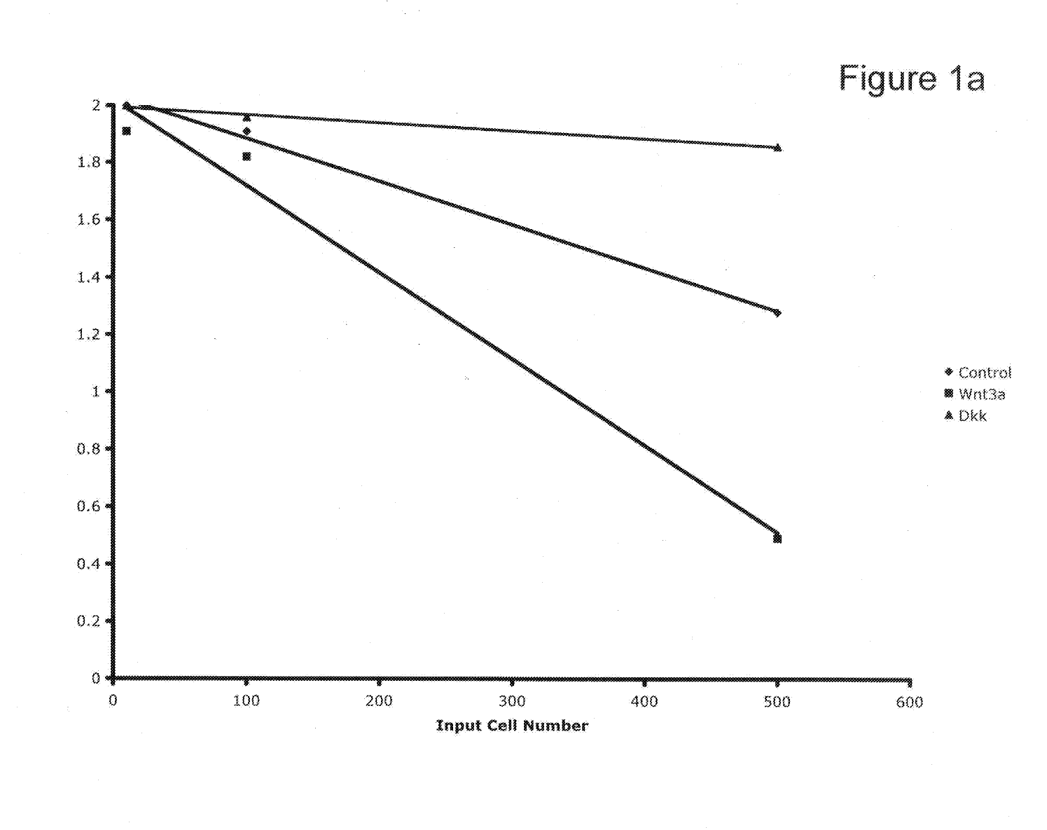

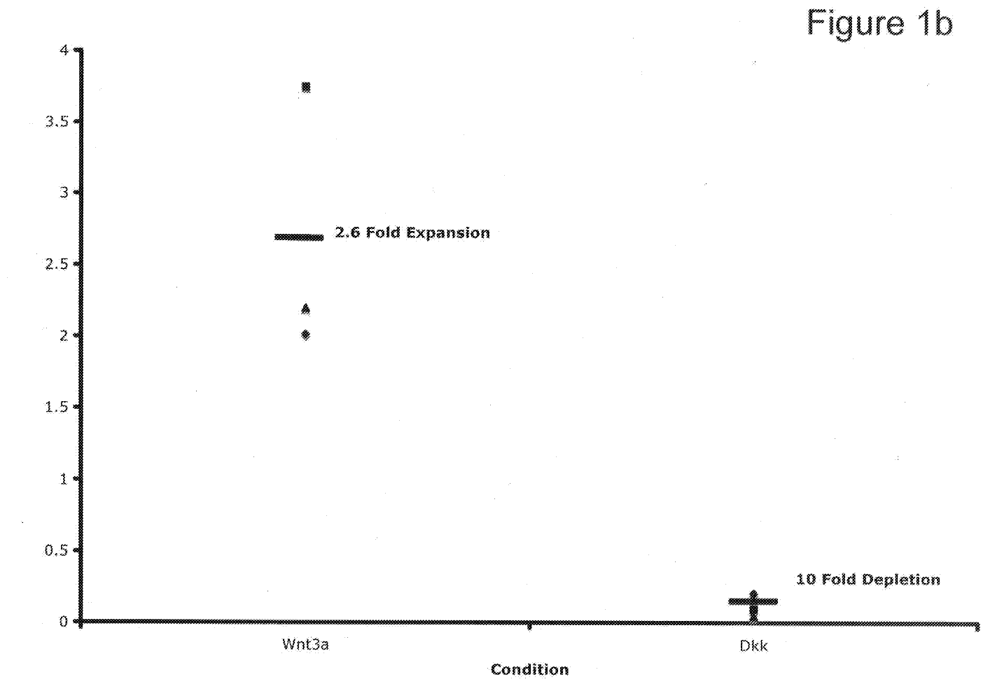

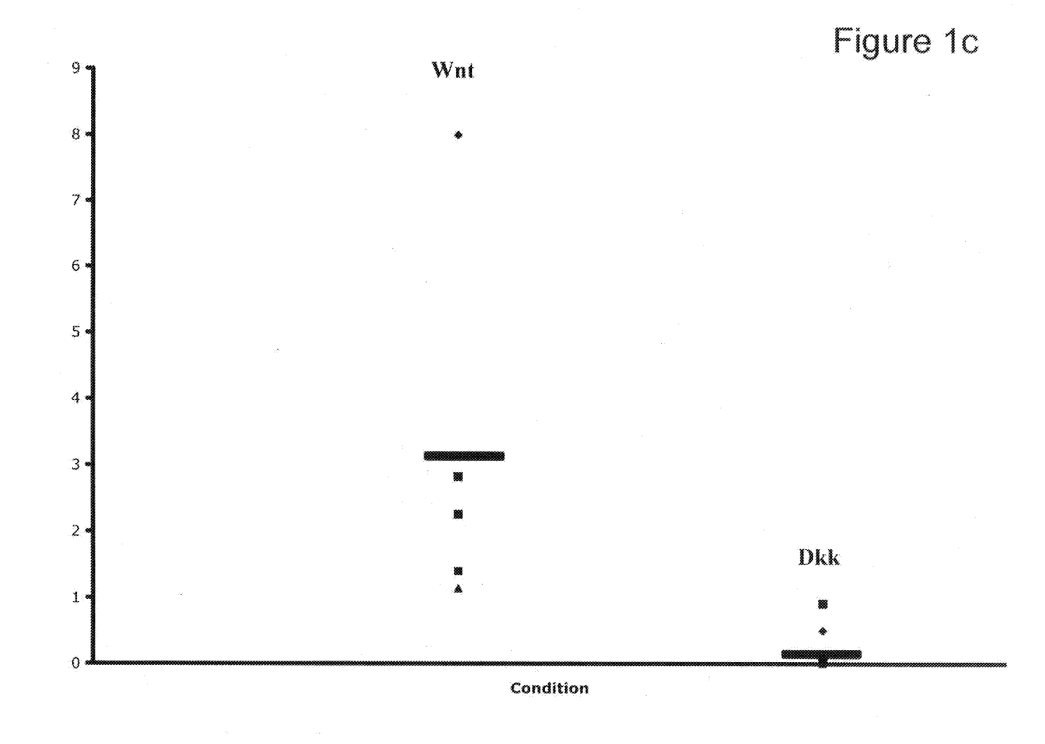

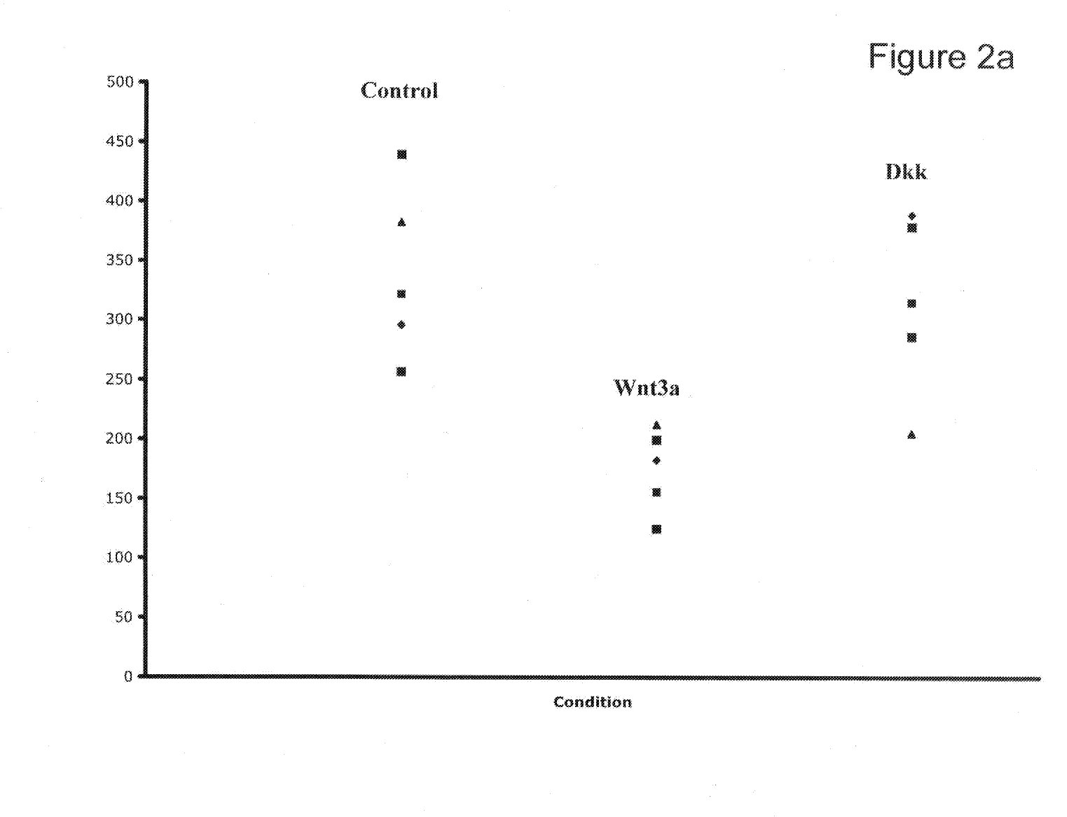

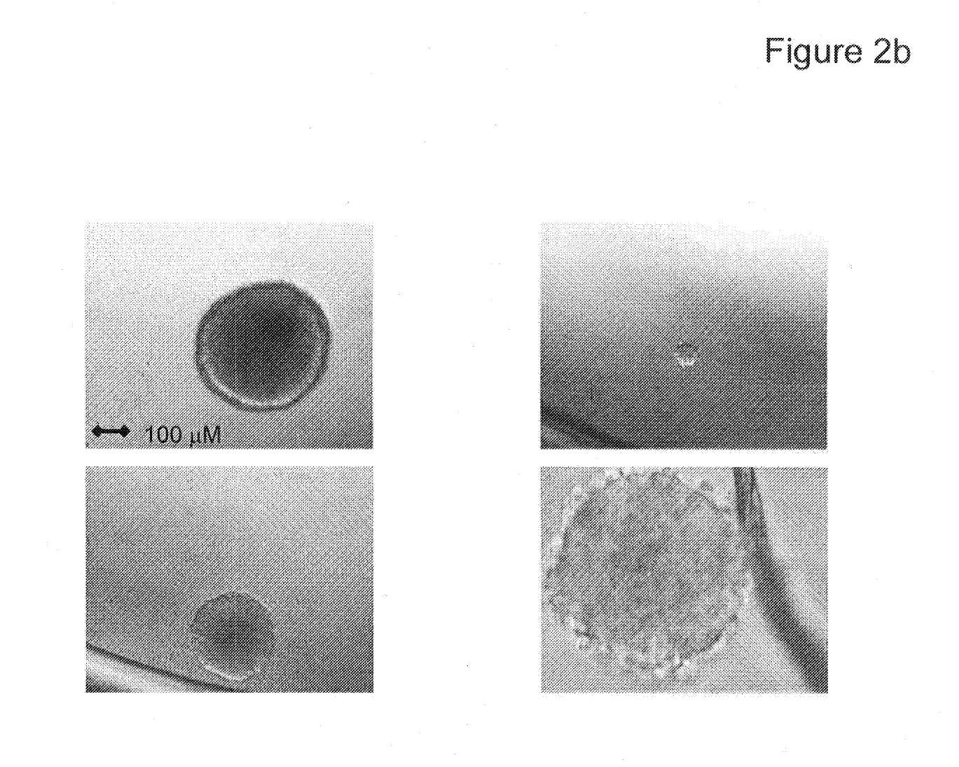

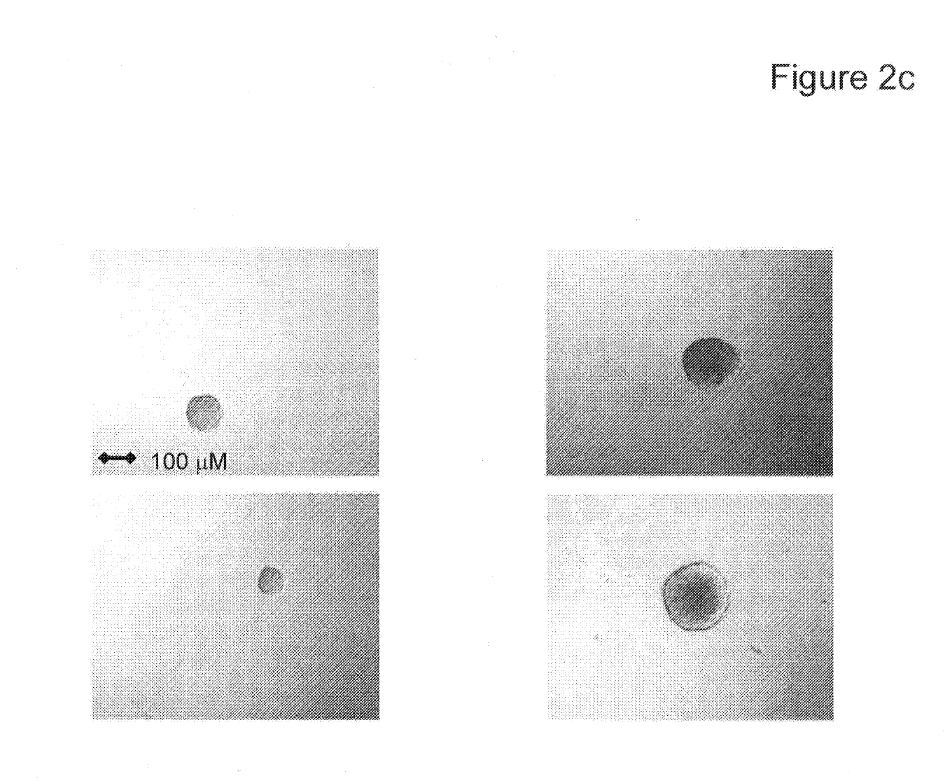

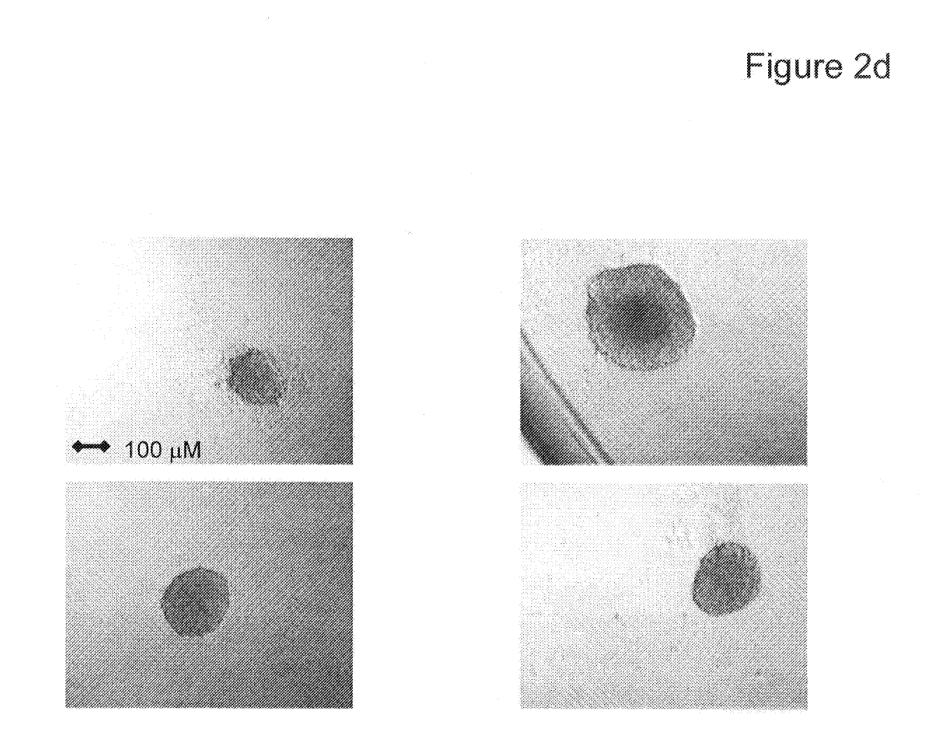

Addition of Wnt protein results in the generation of morphologically unique primary colonies. Using a limiting dilution analysis we plated cells at three cellular densities and assayed for the formation of colonies in the presence of Wnt and Dkk proteins. The addition of Wnt protein generated a greater number of colonies at all cell densities and the Dkk protein resulted in the generation of less number of colonies at all cell densities relative to the control condition (FIG. 1a-c). The neural stem cell initiating colony (NS-IC) is a measure of the stem cells present in the culture of cells plated. Using this value derived from the limiting dilution analysis, it is possible to determine the affect of a growth factor on the maintenance and expansion of stem cells. The NS-IC range, as determined from the limiting dilution analysis shows an average of 2.5-fold expansion over the control with the Wnt, from a NS-IC range of 1/22-1/1490 to 1/10-1/740. The addition of the Dkk results in a decrease in the NS-IC of greater than 10-fold over the control from the range of 1/22-1/1490 to 1/479-1/7321. Morphologically the Wnt and Dkk treated colonies are different. Wnt treated cells generate colonies that are morphologically smaller and more uniform than the colonies treated with mock or Dkk. The Wnt treated colonies generally range in size from 80-250 Meters, nearly half to a quarter of the size of the average vehicle treated colony. The Dkk colonies are uniformly large with a general range of 200-800 Meters (FIG. 2a-d).

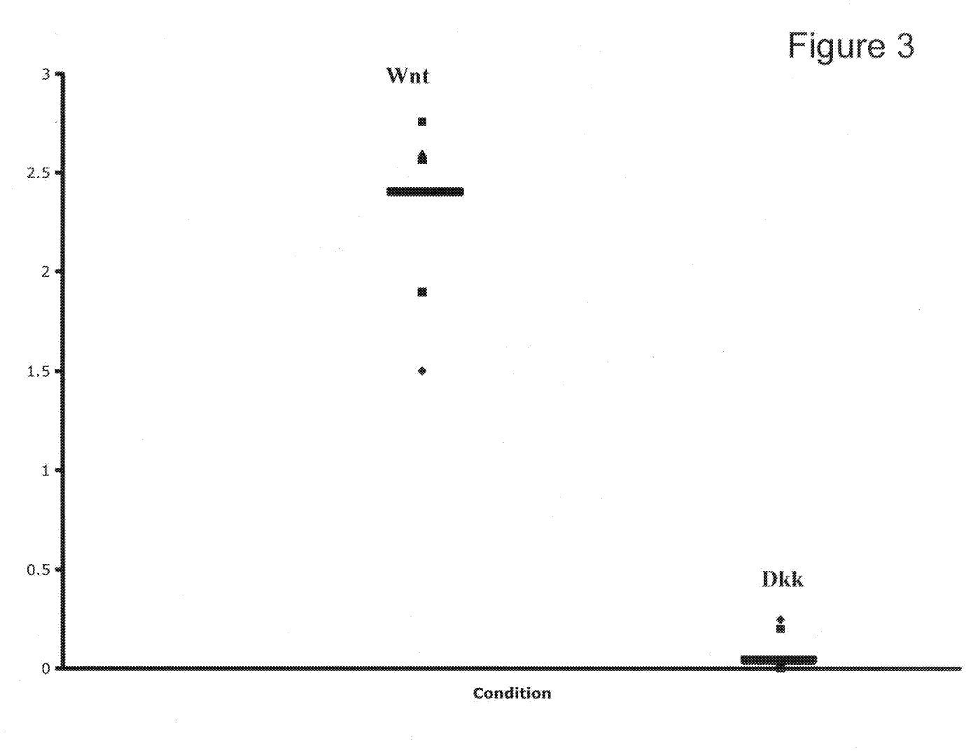

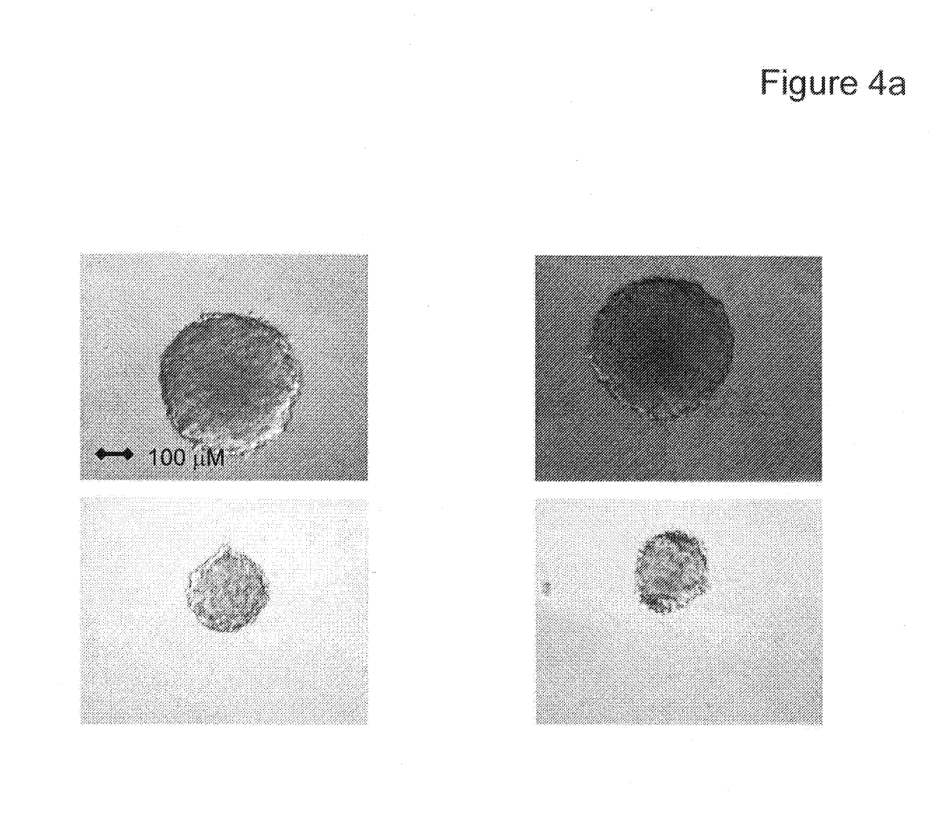

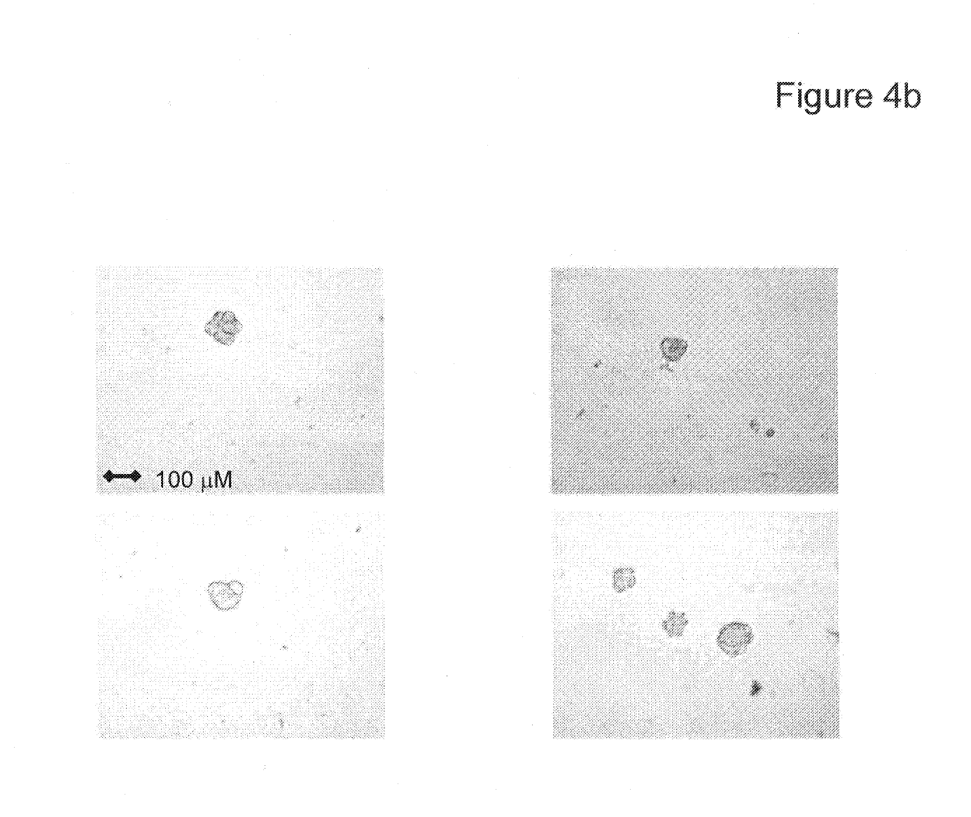

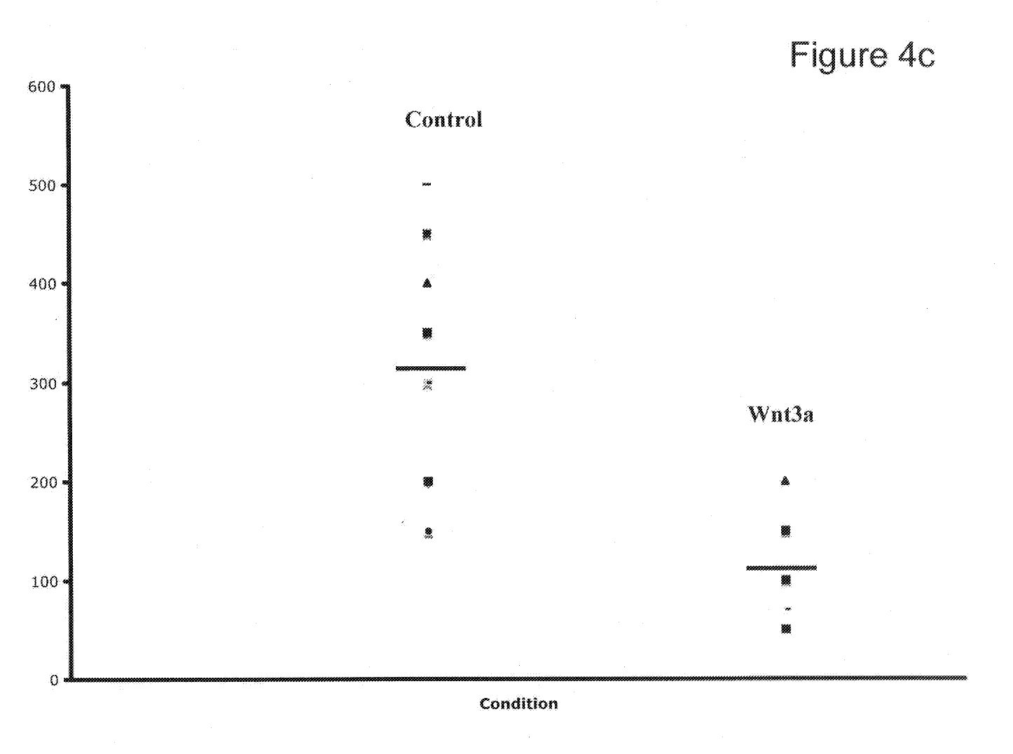

Addition of Wnt protein results in clonogenic expansion of the primary colonies upon re-plating. Cells resulting from the primary colonies were re-plated in the presence of Wnt and Dkk and the ability of these cells to regenerate second colonies in the presence of the factors was assayed. Clonogenic expansion or self-renewal was assayed as a measure of 2° colonies generated per input cell of the 1° colony. The cells cultured in the presence of Wnt resulted in an average clonogenic expansion of nearly 2.5-fold (range of 1.5 to 2.7 fold expansion) relative to vehicle treated stem cell populations, while the cells treated with Dkk showed an, in general, an inability to form secondary colonies (FIG. 3). Again, morphologically the Wnt and Dkk colonies were distinct with the Wnt treated colonies exhibiting a distinct small and morphologically homogenous phenotype (ranging in size from 50-200 μm), while the Dkk treated cultures formed very few large colonies that could not be re-plated a third subsequent time. The vehicle treated colonies formed ranged in size from nearly 100-500 μm (FIG. 4a-c).

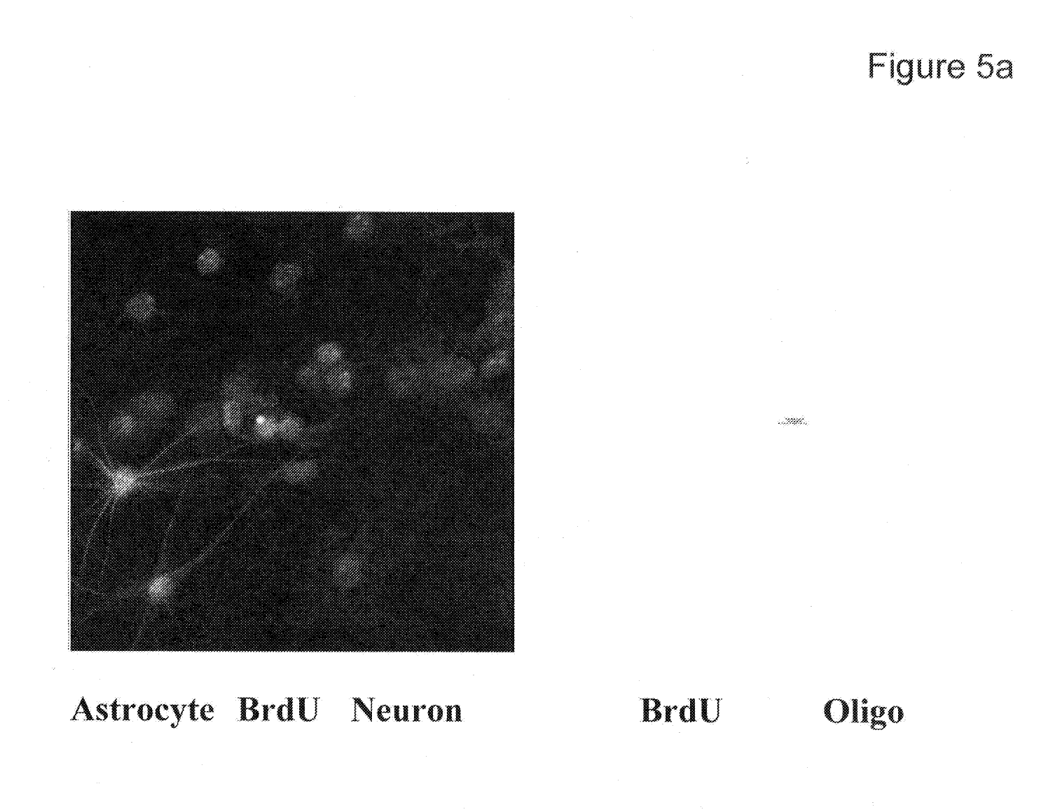

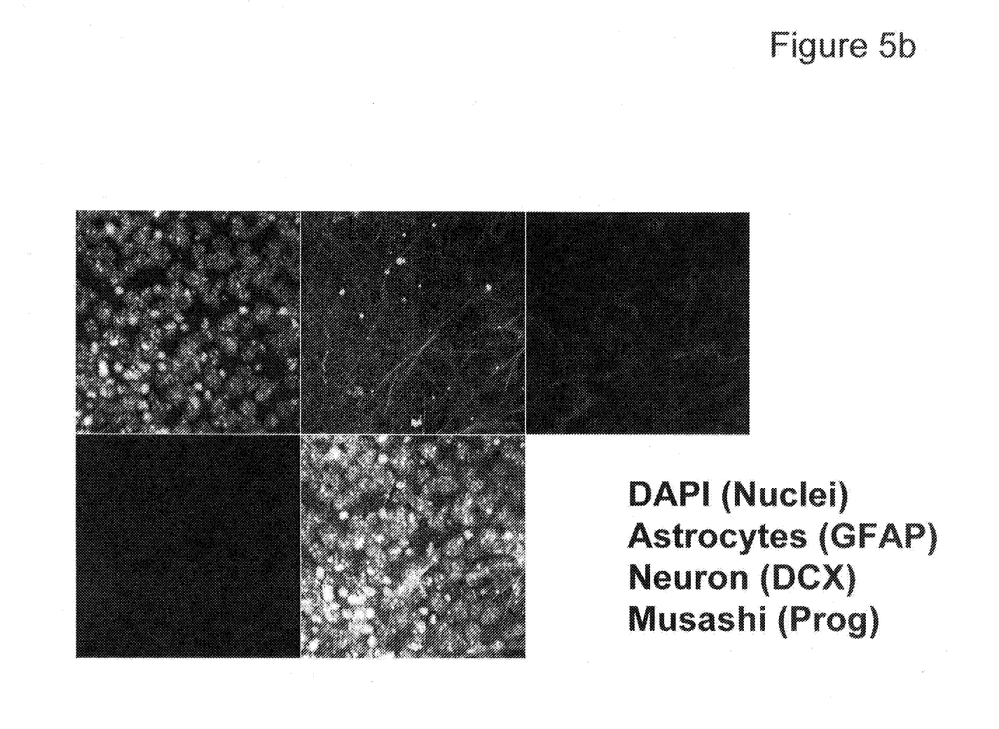

Wnt treated colonies produce colonies that are multipotent. To ensure that the colonies generated as a result of treatment with the soluble Wnt protein are indeed multipotent precursors of the central nervous system we differentiated these colonies for a week in the presence of BrdU, and stained with various progenitor and differentiation markers. Cells treated with the Wnt protein are indeed capable of generating the three cell types of the central nervous system as shown by staining for neuron, oligodendrocyte and astrocytes markers (FIG. 5a-b). The BrdU was added to ensure that the cells generated were indeed born in culture from stem cell populations that existed prior to differentiation by our protocol.

### Discussion

In this study we showed the first application of purified Wnt protein to the in vitro manipulation of neural stem cells. The use of purified protein to study the signaling hierarchy as well as understanding the manipulations necessary to form specific cell types has great applications to the field of cell based therapeutics. Herein we showed that there is a small subpopulation of cells lining the neurogenic zone (SVZ) in the developing mouse brain that is Wnt-responsive. We further note that these cells are radial glial in morphology with foot processes lining the SVZ. These cells further do not stain with the 2 well-established progenitor markers for the CNS (nestin and musashi).

We extended our analysis to the adult brain and observed activation of the signaling pathway in known neurogenic zones in the adult brain, with the AxinII/LacZ homozygous adult brain exhibiting hypercellularity at various neurogenic sites in the brain. We established that a cellular density of 1 or less cells/μl is the cell density producing clonogenic outgrowth and not aggregation of populations in this assay.

Isolation of cells from the forebrain of E14.5 mice and culture in the presence of factors results in a clonogenic outgrowth observed with the Wnt protein. The blockage of the Wnt signal using purified Dkk protein results in a depletion of stem cell and inability to form secondary colonies. The cells treated with the various purified protein are able to differentiate into the cell types of the nervous system, notably neurons, oligodendrocytes and astrocytes. The colonies formed in the presence of factors are not morphologically identical. Wnt treated colonies are small and more homogenous; Dkk treated colonies are larger and more non-homogenous.

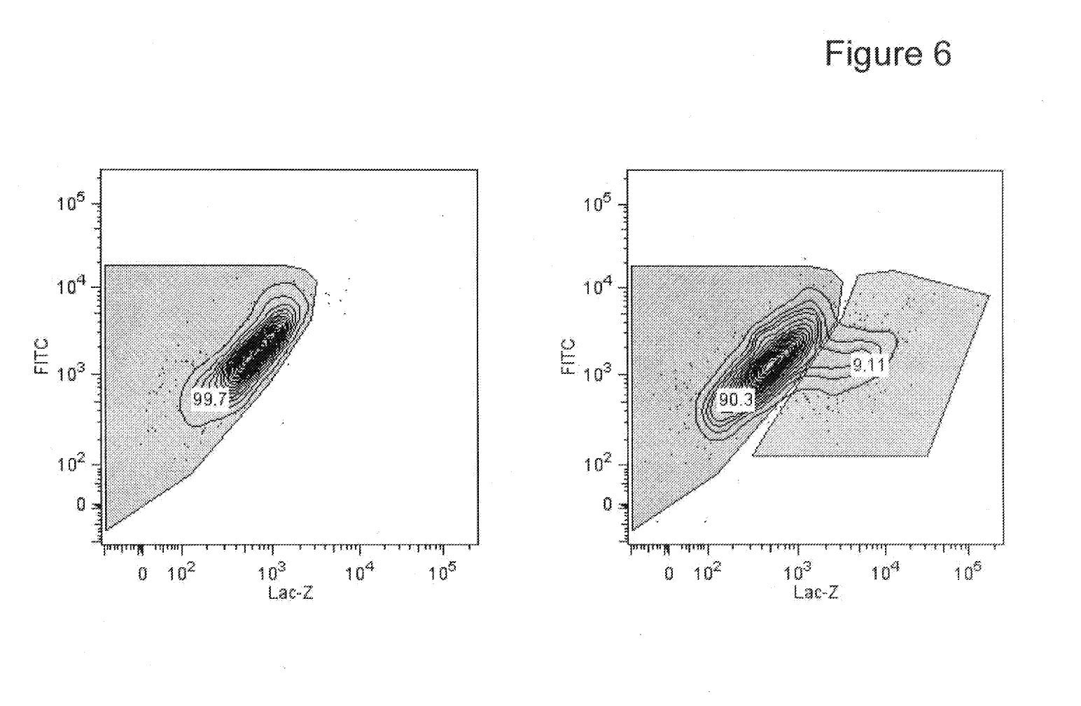

The Wnt-responsive cells lining the SVZ can be isolated using FACS (FIG. 6) and cultured in the presence of the various proteins. The Wnt-responsive cells are morphologically similar to the mixed population of cells when treated with Wnt. The blue cells expand robustly when exposed to the Wnt protein and addition of Dkk prevents the expansion of these cells in culture.

Our observations demonstrate that Wnt signaling is an important pathway in the developing nervous system. The observation that the pathway is active at established neurogenic zones, the ability of the factor to result in expansion of cells in vitro and the observation that blockage of the pathway results in depletion of multipotent cells in culture point to the importance of this signaling pathway as a self-renewing pathway for the central nervous system stem cells. We do not note a mitogenic effect of the purified Wnt protein in vitro; this does not preclude the possibility that this factor may be mitogenic when administered in combination with other factors. The ability to isolate Wnt responsive cells using FACS and manipulate these cells in culture with essential signaling molecules allows for a strategy to both dissect niche effects and selectively manipulate cells in culture to produce selective cell profiles useful in cell transplantation and other therapies.

What is claimed is:

1. A method for in vitro expansion of mammalian neural stem or progenitor cells, the method comprising:
culturing the neural progenitor or stem cells in medium comprising one or more wnt polypeptides;
wherein the number of cells having the functional phenotype of said stem or progenitor cells is expanded.

2. The method of claim 1, wherein the stem or progenitor cell is a stem cell.

3. The method of claim 2, wherein the stem cell is a neural crest stem cell.

4. The method of claim 2, wherein the stem cell is a fetal neural stem cell.

5. The method of claim 2, wherein the stem cell is an adult neural stem cell.

6. The method of claim 1, where the wnt polypeptide is a mammalian wnt polypeptide.

7. The method of claim 6, where the wnt polypeptide is human wnt3A.

8. The method of claim 1, further comprising the step of culturing the neural stem cells after expansion in differentiation medium.
